(19) United States
(12) Patent Application Publication
(10) Pub. No.: US 2014/0212500 A1
(43) Pub. Date: Jul. 31, 2014

(54) COMPOSITIONS CONTAINING DIVERSE NATURAL ANTIGENS AND USES THEREOF IN BALANCING IMMUNE RESPONSES

(71) Applicant: Tse Wen Chang, Taipei City (CN)

(72) Inventor: Tse Wen Chang, Taipei City (CN)

(73) Assignee: Academia Sinica, Taipei (CN)

(21) Appl. No.: 14/239,924

(22) PCT Filed: Aug. 23, 2012
(86) PCT No.: PCT/CN2012/080506

§ 371 Date: Feb. 20, 2014

Related U.S. Application Data
(60) Provisional application No. 61/526,421, filed on Aug. 23, 2011.

(51) Int. Cl. A61K 39/385; A61K 39/00; A23L 1/30; A61K 9/14

(52) U.S. Cl. CPC A61K 39/385; A61K 9/14; A61K 39/0002; A61K 39/0003; A61K 39/00; A23L 1/30
USPC 424/489; 424/184.1; 424/275.1; 424/274.1; 424/265.1; 422/22

(57) ABSTRACT

Compositions containing sterilized antigens with a high diversity, which can be collected from primitive jungle areas, and uses thereof for balancing immune responses and treating immunological diseases in a subject.

### RELATED APPLICATION

This application claims the benefit of U.S. provisional application No. 61/526,421, filed Aug. 23, 2011, the entire content of which is incorporated by reference herein.

### BACKGROUND OF THE INVENTION

Allergic diseases, including allergic asthma, allergic rhinitis, atopic dermatitis, and food allergy, are characterized by a hypersensitivity of a patient's immune system to harmless environmental substances (known as allergens). IgE antibodies play an important role in allergy. Induced by allergens, IgE activates mast cells and basophils by interacting with its high-affinity receptor FcεRI expressed on these immune cells, resulting in excessive inflammatory responses, which lead to allergic diseases.

Autoimmune diseases, such as rheumatoid arthritis, systemic lupus erythematosus, multiple sclerosis, Type I diabetes, and inflammatory bowel disease, are characterized by abnormal immune responses attacking autologous (self) antigens, causing tissue damage. It has been suggested that IgE antibodies might also contribute to autoimmune responses. Autologous antigens may induce the production of IgE, as well as IgG autoantibodies, which augment inflammation. Deregulation of T regulatory cells is also found to contribute to the development of autoimmune diseases.

Allergic and autoimmune diseases become more and more prevalent in modern society, indicating that urbanized living environment may play a role in the development of these immune disorders. Current anti-allergy and anti-autoimmune therapy include antagonistic drugs, such as antihistamines and anti-leukotrienes, anti-inflammatory drugs such as corticosteroids, desensitization therapy with allergy shots, anti-IgE antibodies such as Omalizumab, and specific immunosuppressants such as anti-CD20 and anti-tumor necrosis factor antibodies. Given the prevalence of these immune diseases, it is of great interest to develop new therapeutic approaches targeting these diseases.

### SUMMARY OF THE INVENTION

The present disclosure is based on the idea that exposure to foreign antigens with a large diversity balances immune systems and is effective in treating immune disorders such as allergic diseases and autoimmune diseases.

One aspect of the present disclosure relates to a composition comprising a mixture of sterilized antigens, which is prepared by a process comprising: (i) placing a particle collector in a jungle area, (ii) harvesting airborne antigens to obtain a mixture of harvested airborne antigens, and (iii) sterilizing (by, e.g., gamma or X ray irradiation) harvested airborne antigens, thereby obtaining the mixture of the sterilized antigens. Optionally, non-biological substances are removed from the harvested airborne antigens via, e.g., density gradient centrifugation, preferably prior to sterilization.

When desired, the particle collector collects substantially only airborne antigens having a diameter ranging from about 0.5 μm to about 200 μm (e.g., 0.5 μm to 150 μm or 0.5 μm to 100 μm). The airborne antigens can be harvested from the particle collector 1 to 7 days after the collector was placed in the jungle area. This harvesting process can be repeated for up to 12 months (e.g., 1 month, 3 months, 6 months, or 9 months) and, when necessary, the total airborne antigens can be combined. In one example, the composition contains airborne antigens collected in one day (daily packs). In other examples, the composition contains antigens collected in one week (weekly packs), one month (monthly packs), or one season (seasonal packs).

The composition can be a food product, or a dietary supplement, or a pharmaceutical product, which further comprises a pharmaceutically acceptable carrier. It can be formulated for non-invasive administration, e.g., intranasal administration (spray or inhaler), sublingual administration, or oral administration.

In another aspect, the present disclosure provides kits for balancing immune responses (e.g., enhancing production of diverse IgE antibodies or balancing activity of T regulatory cells), treating an allergic disease, and/or treating an autoimmune disease. Each kit comprises multiple preparations of sterilized antigen mixtures (e.g., at least 4 preparations such as 4-8 preparations, 4-12 preparations, or 4-24 preparations). These multiple preparations can be pharmaceutical products, which contain one or more pharmaceutically acceptable carriers, food products, or dietary supplements. Preferably, they are formulated for non-invasive administration, such as intranasal administration, sublingual administration, or oral administration.

In some embodiments, the sterilized antigen mixture in each of the multiple preparations in the kit is prepared by the process described above, each preparation containing airborne antigens collected from a jungle area. The sterilized antigen mixtures in different preparations are obtained from geographically different jungle areas or from the same jungle area but during different time periods over a year (e.g., different seasons) or in different years. The airborne antigens can have a diameter ranging from about 0.5 μm to about 200 μm (e.g., 0.5 μm to 150 μm or 0.5 μm to 100 μm). Each preparation can contain antigen mixtures collected (once or multiple times) in a jungle area during a suitable period (e.g., up to 12 months, such as 1 day, 1 week, 1 month, 3 months, 6 months, or longer). In one example, the kit contains one or more daily packs described above. In other examples, the kit contains one or more of the weekly packs, monthly packs, or seasonal packs.

In other embodiments, the multiple preparations in the kit each comprise at least five sterilized natural antigens (e.g., weed pollens, tree pollens, mold antigens, microorganism antigens, or worm antigens), the amount of each of which is effective in inducing IgE antibody production. At least 20% of the natural antigens in one preparation are not present in any other preparations in the kit. The natural antigens can be sterilized by gamma ray or X irradiation. In one example, these antigens are geographically specific. Such antigens are abundant in a specific geographical area and their abundance in other geographical areas is substantially less (e.g., 50% less, 100% less, 2-fold less, 5-fold less, 10-fold less, 20-fold less, 50-fold less, 100-fold less, or 1000-fold less). In some examples, a geographically specific antigen is present only in that specific geographical area.

The present disclosure also provides a method for enhancing IgE antibody production using any of the compositions/kits disclosed herein. In some embodiments, the method comprises administering to a subject in need thereof an effective amount of any of the compositions described above that comprise a mixture of sterilized antigens. The composition can be delivered via a non-invasive route, such as intranasal administration, sublingual administration, or oral administration. In one example, a subject is exposed to the sterilized antigens by staying in a room (e.g., home, office, or daycare center) where the mixture of sterilized antigens is released.

In other embodiments, the method comprises exposing a subject in need thereof sequentially the multiple preparations in any of the kits disclosed herein via, e.g., a non-invasive route as those described herein. Optionally, the subject is exposed to each of the multiple preparations for 1 to 4 weeks.

The subject to be treated by any of the methods disclosed herein can be a human patient having, suspected of having, or at risk for an allergic disease (e.g., allergic asthma, allergic rhinitis, atopic dermatitis, or food allergy) or an autoimmune disease (e.g., rheumatoid arthritis, systemic lupus erythematosus, multiple sclerosis, Type I diabetes, autoimmune thyroid disease, or inflammatory bowel disease).

Also disclosed herein are compositions and kits as described herein for use in enhancing IgE antibody production, balancing immune responses (e.g., balancing T regulatory cell activities), treating an allergic disease, and/or treating an autoimmune disease, as well as use of any of the compositions for manufacturing a medicament for any of the just-noted purposes.

The present disclosure further provides a method for preparing a composition that comprises a mixture of sterilized antigens. The method comprises:

(i) placing a particle collector in a jungle area,

(ii) harvesting airborne antigens collected in the particle collector, and

(iii) sterilizing (e.g., by gamma or X-ray irradiation) the airborne antigens to obtain a composition, which comprises a mixture of sterilized antigens.

Optionally, the above-described method further comprises removing non-biological substances from harvested airborne antigens by, e.g., density gradient centrifugation.

The particle collector can be adjusted such that it collects substantially only airborne antigens having a diameter from about 0.5 μm to about 200 μm (e.g., 0.5 μm to 150 μm or 0.5 μm to 100 μm). The airborne antigen can be harvested from the particle collector 1 to 7 days after the collector was placed in a jungle area. When necessary, the airborne antigens are harvested from a particle collector once every 1 to 7 days for up to twelve months. The antigens thus collected can either be packed individually or combined. In one example, airborne antigens are collected in a jungle area each day and each daily collection is processed and packed individually. In other examples, airborne antigens are collected daily and those collected in one week, one month, or one season are combined for further process and package.

The preparation method can further comprise formulating the composition to produce a pharmaceutical product, a food product, or a dietary supplement.

The details of one or more embodiments of the invention are set forth in the description below. Other features or advantages of the present invention will be apparent from the following drawing, detailed descriptions of several embodiments, and also from the appended claims.

### BRIEF DESCRIPTION OF THE DRAWINGS

The drawings are first described.

FIG. 1 is a photo showing the SDS PAGE analytical patterns of protein in airborne particles collected in various locations. The protein in filters was dissolved in PBS with 0.1% Tween 20 and run on a 13% SDS PAGE and stained with Pierce Silver Stain Kit. Lane 1, blank filter; lane 2, bedroom in a home (2012 Apr. 4); lane 3, the P.I.'s research laboratory (2012 Aug. 15) in the Genomics Research Center (GRC), Academia Sinica; lane 4, the P.I.'s office in GRC (2012 May 20); lane 5, Fu-Shan National Park (2012 Jun. 8); lane 6, covered hallway outside GRC (2012 May 2); lane 7, Yuan-Pei Park in Academia Sinica (2012 Jul. 13); lane 8, Yuan-Pei Park (2012 Jul. 26); lane 9, Yuan-Pei Park (2012 Aug. 9); lane 10, Yuan-Pei Park (2012 Jul. 13), from the 2nd extraction of filter; lane 11, Yuan-Pei Park (2012 Jul. 26), from the 2nd extraction of filter; lane 12, Yuan-Pei Park (2012 Aug. 9), from the 2nd extraction of filter; lane 13, bovine serum albumin

### DETAILED DESCRIPTION OF THE INVENTION

Without being bound by theory, the present disclosure is based at least on the idea that unbalanced immune systems contribute to immunological disorders such as allergy and autoimmunity and that exposure to highly diverse foreign antigens promotes restoration of immune balance and is effective in treating immunological disorders or reducing the risk for developing such disorders. Foreign antigens include antigens derived from an area where a subject has not lived for more than two years or those that a subject has not been exposed to.

Allergic and autoimmune diseases are more prevalent in human populations living in an urbanized environment than those living in a primitive environment. People living in a primitive habitat have the opportunity to be exposed to a large number of highly diverse natural antigens in their living environment (e.g., pollens of local flora, which change over time), particularly in their infancy and childhood. No particular antigen is dominant in the whole natural antigen pool to which they are exposed. As such, these people are more likely to establish balanced immune systems, which is characterized by the presence of a highly diverse IgE antibody population or balanced T regulatory cell activities. In that case, the FcεRI receptors on mast cells and basophils are not occupied by a large amount of IgE antibodies specific to certain allergens/autologous antigens; thus, no excessive inflammatory responses against those allergens/autologous antigens, which cause allergy or autoimmunity, would be triggered.

By contrast, people living in urbanized environment have fewer infections in infancy and childhood and are primed with fewer foreign antigens, which results in an under-stimulated TH1 pathway and an unleashed TH2 pathway. The enhanced TH2 activities promote production of inflammatory cytokines, such as IL-4 and IL-6, and consequently, inducing IgE antibodies targeting environmental allergens and/or autologous antigens. Patients who are allergic to certain allergens are found to have skewed IgE antibody populations specific to those allergens. That is, IgE antibodies specific to the allergens account for high proportions of the total IgE antibody pool. These IgE antibodies activate mast cells and basophils, triggering excessive inflammatory responses against the allergens (or autologous antigens), leading to development of allergy and autoimmunity. Chang et al., “Cumulative environmental changes, skewed antigen exposure, and the increase of allergy.;” Adv. Immunol. 98, 39-84 (2008).

The present disclosure aims at maintaining and/or restoring immune balance in a subject by exposing the subject to sterilized natural antigen mixtures with high diversities. These diversified antigens can stimulate production of a balanced IgE antibody pool and/or balanced T regulatory cell activities in the subject (a subject who has IgE antibodies specific to a large variety of antigens). In that case, IgE antibodies specific to dominant local antigens present in the place where the subject lives constitute only a very small portion of the total IgE antibody pool. Stimulation of a subject's immune system with diverse antigens also enhances other immune components, such as IgG and IgA against those natural antigens. Presence of these immune components can neutralize and eliminate the antigens that get into the subject via inhalation and ingestion.

Accordingly, described herein are compositions containing sterilized natural antigens (e.g., those collected from jungle areas), methods for preparing such, and uses thereof for balancing immune systems (e.g., inducing production of diverse IgE antibodies or balancing T regulatory cell activity), treating allergic diseases, or treating autoimmune diseases.

Preparation of Sterilized Antigen Mixtures from Jungle Areas

The natural antigens to be used in balancing immune systems as described herein can be collected from jungle areas using a particle collector (also known as dust collector).

### Jungle Areas

Jungles areas, particularly primitive jungle areas, contain natural antigenic substances that most human beings (e.g., those living in urbanized places) have not been exposed to. These are preferred places for harvesting natural antigens (antigens present in nature or derivatives thereof). Preferably, a jungle for natural antigen collection is away from a human habitat for at least 20 km.

A jungle area can be a bio-diverse and uncultivated thicket wherein plants grow naturally. They can be located at areas far away from human habitats and have no human activities (e.g., primitive jungles). There should not be artificial planting in the areas where the natural airborne particles are to be collected.

One example of jungle areas are rainforests, including tropical rainforests found in the equatorial zone (between the Tropic of Cancer and Tropic of Capricorn) and temperate rainforests. Tropical forests include, but are not limited to those present in Southeast Asia (from Myanmar (Burma) to Philippines, Indonesia, Papua New Guinea and northeastern Australia), Sri Lanka, sub-Saharan Africa from Cameroon to the Congo (Congo Rainforest), South America (e.g. the Amazon Rainforest), Central America (e.g. Bosawás, southern Yucatan Peninsula-El Peten-Belize-Calakmul), and on many of the Pacific Islands (such as Hawaii). Temperate rainforests occur in North America (in the Pacific Northwest, the British Columbia Coast and in the inland rainforest of the Rocky Mountain Trench east of Prince George), in Europe (parts of the British Isles such as the coastal areas of Ireland and Scotland, southern Norway, parts of the western Balkans along the Adriatic coast, as well as in the North West of Spain and coastal areas of the eastern Black Sea, including Georgia and coastal Turkey), in East Asia (in southern China, Taiwan, much of Japan and Korea, and on Sakhalin Island and the adjacent Russian Far East coast), in South America (southern Chile) and also in Australia and New Zealand. Other Exemplary jungle areas include, but are not limited to, forests of northern Thailand or southern Guangdong in China, Xishuangbanna, Yunan Province, China, and forests in Ping-Dong, Taiwan.

### Particle Collector

Airborne particles in a jungle area can be collected using a particle collector, which can be any device or equipment suitable for filtering airs and harvesting particles contained therein, including those that utilize centrifugal, fabric filters (baghouse), or wet scrubber mechanisms. If the particle-collecting device employs a wet scrubbers mechanism, all retained substances, including protein molecules released from trapped particles, are included for further processing.

Particle collectors are commonly used in industry for collecting valuable particles in process stream in a manufacturing process chain or in removing harmful particulates from exhaust gases prior to venting the gases to the atmosphere. Some of those are installed in site and not readily movable, while others are movable and can be carried or transported to various locations. Many portable devices have been designed and manufactured for use in collecting airborne particles in large volumes of air, for example, pollen particles and human, animal, and plant pathogens for allergy, medicine, agriculture, or counter-terrorism monitoring.

A particle collector suitable for use in the methods described herein can employ filter mesh with a very large surface for catching the airborne particles based on built-in electrical field. In this type of device, as the incoming particles impact on the surface in the filter, electrical charges are generated on the particles and on the mesh surface and hence the particles become trapped. Some of these devices, which have a capacity to filter a few thousand liters of air per minute, are small and portable, and run by batteries and can be used in remote outdoor areas. The opening of the particle-collecting device faces somewhat downward or the device can be protected by an umbrella-like shield above it, so that it can continue to operate and collect airborne particles in a rainy condition. The air is channeled into the device by the aid of a motor.

Various types of particle collectors are commercially available, including the SASS serials (e.g., SASS 4000, SASS 4100, and SASS 2300) provided by Research International, Inc. (Monroe, Wash., USA) and the Biral Aerosol Particle Collectors provided by Biral APC, UK.

Typically, a particular collector for use in collecting airborne particles is a highly efficient, high-volume aerosol concentration device suitable for collecting airborne articles having desired diameter ranges. It can process a large volume of ambient air and continuously transfer particulates from this primary air stream to a much smaller secondary airflow. As a result, the secondary flow can reach aerosol concentrations that are several folds higher than present in the incoming air. The concentrator therefore amplifies the ambient aerosol concentration, while retaining most of the particles that were present in the incoming airflow in the secondary flow. Particles are routed into the secondary flow by forcing primary circuit air to circulate through specially shaped channels where centrifugal force and particle momentum are used to isolate and concentrate the particles. The interior structure can be designed so that the smallest flow cross-section is a channel of desired sizes, providing good resistance to clogging by larger particles.

A particle collector to be used in harvesting airborne antigens can be adjusted such that is only collects airborne particles having a pre-determined diameter range. For example, the collector can contain a coarse screened cover (mesh) at the place where ambient air enters to restrict the entrance of large debris. When necessary, a particular collector having a tamper proof design can be adopted for outdoor use in sub-zero temperatures.

### Airborne Particle Collection and Processing

A particular collector can be placed in a jungle area. In one example, the collector is adjusted for collecting substantially (at least 70% of the total collection) airborne particles have diameters ranging from 0.5 μm to 200 μm (e.g., 0.5 μm to 150 μm, or 0.5 μm to 100 μm). This allows collecting different types of airborne particles in the jungle area. Typically, airborne particles contain pollen particles of grasses, weeds, trees, spores of bacteria and fungi as single cells and aggregate forms, protozoa, animal danders, fine shed or broken parts of plants and small animals, and other substances derived from plants or animals. The pollen particles from most plants are in the range of a few μm to 100 μm and very few of them are larger than 100 μm. The spores of various origins are in the range of 1 μm to 20 μm. The smallest insects, such as fairy flies and some feather-winged beetles, have lengths or wing spans of about 200 μm.

Airborne antigens collected by the particle collector can be harvested after the collector has been placed in the jungle area for a predetermined period (e.g. 1 to 7 days) depending upon, e.g., the capacity of the collector. This process can be repeated for up to twelve months to collect different antigens present in different time periods over a year or in different years. Antigens in each collection can be packed and processed individually (i.e., single-day packs). Alternatively, antigens in several collections can be combined for further process (e.g., weekly packs, which include antigen particles collected in 7 days, monthly packs, which include antigens collected in one month, and seasonal packs, which include antigens collected in three months). In one example, antigens are collected in a jungle area every day for a suitable period of time (e.g., 6 months, 12 months, or 24 months). The daily collections can be processed and packed individually to form daily packs. Alternatively, those collected in one week, one month, or one season can be combined for processing and packaging to produce weekly, monthly, or seasonal packs.

Airborne particles often contain non-biological substances (substances not derived from organisms) such as fine dirt particles and fine sand particles. These non-biological substances are often heavier than biological particles and therefore can be removed by routine methods, such as density gradient centrifugation (e.g., sucrose or glycerol density gradient centrifugation). Upon the exposure or immersion into liquid medium, the antigen particles, such as pollens, undergo partial breakdown and release some of their proteins in the liquid medium. Thus the enriched fraction should contain intact and partially broken particles and released protein molecules.

The natural antigens collected from a jungle area can then be dried and sterilized by routine technology, such as gamma ray or high-energy X ray irradiation. Sterilization minimizes any kind of infection or unnecessary spread of plant and animal species, especially across geographical regions or countries.

Typically, the natural antigen mixtures prepared as described above are highly diverse and contain various antigens derived from, e.g., weeds and trees (e.g., from pollens), antigenic substances from microorganisms, small insects, and worms, antigenic substances from spores of fungi and bacteria, antigens from mites, and antigenic substances from animals.

The natural antigens thus collected can be characterized by routine technologies, including (1) traditional morphological and taxonomical methods, (2) molecular genetic methods, which identify genes encoding the proteins of interest and analyze the DNA coding sequences, and (3) immunochemical methods, which analyze the antigenic cross-reactivity with known allergens. These antigens can be examined to determine cross-reactivities with a known allergen using the IgE antibody assay described in Example 1 below.

### Antigen Packages Containing Multiple Preparations of Diverse Antigens

Also described herein is an embodiment of packages each containing multiple antigenic preparations (e.g., 4-12 preparations such as 4 or 8), each of which are composed of sterilized antigens (known or characterized) that are prepared or purified individually. A key criterion for designing the immune package is that the antigenic substances in each preparation of a package are of divergent varieties such that they have little or no cross-reactivity and no single antigen accounts for a large proportion in the mixture of antigens. Each preparation can contains at least five (e.g., 10 or 15) characterized antigens each in an amount sufficient to induce IgE antibody production. In one example, no two or more antigenic substances in each preparation co-exist in the same geographical region during the same time period (e.g., the same season). In another example, the immune package contains at least 2 preparations each containing 15 or more antigens.

Preferably, each preparation does not include a dominant antigen. For example, none of the antigens contained therein constitutes more than 50% (e.g., 40%, 30%, 20%, or 10%) of the total antigens in that preparation. In each preparation, at least 20% of the antigens are not present in any of the other preparations in the same package.

The natural antigenic substances, which are functionally suitable for being included in the immune packages noted above include pollens of weeds, pollens of trees, molds, mites, microorganisms, worms (nematodes), and other non-infectious environmental bio-substances. Even dead infectious microorganisms and parts of dead infectious parasites may also be functionally applicable. Preferred natural antigenic substances include, but are not limited to, pollens of weeds of divergent species, pollens of trees of divergent species, and molds of divergent species. In one example, the immune packages include natural antigenic substances that require minimal manufacturing processing and retain the natural properties.

Antigenic substances used in preparing the immune packages noted above should exclude those that are distributed in various geographical areas. Non-preferred antigenic substances include allergens in food (e.g., peanuts, tree nuts, dairy products, wheat proteins), household animal antigenic substances (e.g., danders of cats and dogs), antigenic substances from farm animals and cultured fishes, and antigenic substances from agricultural crops and plants, such as peanut, wheat, rice, soy beans. Similarly, trees having related species growing in various geographical regions for fruits, lumber, shades, wind shield, ornamental, and other purposes and grasses growing in many geographical regions for ground covering are also not preferred sources for the natural antigenic substances to be included in the immune packages.

By contrast, species of weeds and trees that have no commercial or ornamental values, often grow in limited geographic regions (e.g., geographic specific). Antigenic substances from these plants are preferred antigens to be included in the immune packages. The natural antigenic substances to be included in the immune packages can be collected from geographical regions having distinctive flora as compared to areas with high human densities, such as U.S.A., China, Russia, Canada, India, Indonesia, Brazil, and Europe (e.g., U.K., France, and Germany).

Geographical areas with distinct plant populations are ideal places for obtaining the antigens. For example, it is estimated that 85% of the 12,000 species of flowering plants in Madagascar Island are found nowhere else on earth. Among those plants are many whose pollen antigens should be minimally cross-reactive with the pollen antigens in Japan, the US, and Europe. Another example is Australia, where the spread of weeds of many varieties cause economic problems and do not grown in other countries. Antigenic substances from these weeds can be used for preparing the packages described herein.

The characterized natural antigens to be included in the immune packages can be prepared by isolation from their natural sources or made via routine recombination technology.

### Methods for Balancing Immune Systems

Any of the antigen mixtures, either collected from jungle areas or containing characterized antigenic substances, can be formulated as pharmaceutical composition, food products, or dietary supplements via methods known in pharmaceutical and food industries for any of the utilities described herein. The product may be prepared in the form of powder, liquid suspension, gel, paste, tablets, capsule, or gum for being taken orally (e.g., as drink or food supplement). When the antigenic substances are formulated into a chewing gum, they can be maintained in the mouth and lingual areas for an extended time, allowing their contact with mucosal epithelium.

To prepare pharmaceutical compositions, an antigen mixture can be combined with a pharmaceutically acceptable carrier. The pharmaceutical compositions can be formulated for various routes of administrations, preferably for non-invasive administration, such as intranasal administration, sublingual administration, or oral administration. The carrier in the pharmaceutical composition must be “acceptable” in the sense that it is compatible with the active ingredient of the composition, and preferably, capable of stabilizing the active ingredient and not deleterious to the subject to be treated. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation. See, e.g., Remington's Pharmaceutical Sciences, 16th edition, Mack Publishing Co., Easton, Pa. (1980); and Goodman and Gilman's “The Pharmacological Basis of Therapeutics”, Tenth Edition, Gilman, J. Hardman and L. Limbird, eds., McGraw-Hill Press, 155-173, 2001.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation.

Alternatively, the antigen mixtures can be formulated for parental injections, including subcutaneous, intracutaneous, intravenous, intramuscular, intraarticular, intraarterial, intrasynovial, intrasternal, intrathecal, intralesional, and intracranial injection or infusion techniques. A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol.

To practice the immune balance methods described herein, any of the antigen mixtures can be administered to a subject in need of the treatment at an amount effective in inducing production of IgE antibodies specific to these antigens, balancing immune responses (determined by, e.g., examining the levels of TH1 and TH2 cytokines before and after the treatment, or examining the levels of T regulatory cell activity before and after the treatment), or treating allergic/autoimmune diseases. The term “treating” as used herein refers to the application or administration of a composition including one or more active agents to a subject, who has an allergic/autoimmune disease, a symptom of the allergic/autoimmune disease, or a predisposition toward the disease, with the purpose to cure, heal, alleviate, relieve, alter, remedy, ameliorate, improve, or affect the disease, the symptoms of the disease, or the predisposition toward the disease.

“An effective amount” as used herein refers to the amount of an antigen that alone, or together with further doses or one or more other active agents, produces the desired response, e.g., those noted above. In the case of treating a particular allergic disease or autoimmune disease, the desired response is inhibiting the progression of the disease. This may involve only slowing the progression of the disease temporarily, although more preferably, it involves halting the progression of the disease permanently. This can be monitored by routine methods, such as physical examination and immunological analysis. The desired response to treatment of the disease or condition also can be delaying the onset or even preventing the onset of the disease or condition.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The interrelationship of dosages between animals and humans (e.g., based on milligrams per meter squared of body surface or milligrams per body weight) is well known in the art. See, e.g., Freireich et al., (1966) Cancer Chemother Rep 50: 219. Body surface area may be approximately determined from height and weight of the patient.

A subject to be treated by the method described above can be a human patient suffering from an allergic disease (e.g., allergic asthma, allergic rhinitis, atopic dermatitis, or food allergy) or from an autoimmune disease (e.g., rheumatoid arthritis, systemic lupus erythematosus, multiple sclerosis, autoimmune thyroid disease, Type I diabetes, or inflammatory bowel disease), at risk for, or suspected of having the disease. A patient having a preexisting allergic/autoimmune disease can be identified by a routine medical procedure, e.g., immunological assays. A subject at risk for developing a disease is associated with one or more risk factors for that disease. Risk factors for allergy include cigarette smoke (positive or negative), births during high-pollen seasons, allergen exposure, born prematurally, family history of allergy, age (children are more likely to develop allergy). Risk factors associated with autoimmunity include cigarette smoking, previous infection, family history, and genetic factors associated with autoimmunity. A subject suspected of having an allergic or autoimmune disease might be asymptomatic or show one or more symptoms of the disease, which are well known in the art.

In some embodiments, a subject in need of the treatment is exposed to any of the antigen mixtures described herein via a non-invasive route similar to the route through which the subject is exposed to antigens in his living environment (local antigens). The primary route is to allow the natural antigen mix to contact the respiratory and gastrointestinal tracts of a recipient, including the nasal, oral, ocular mucosal linings. In one example, the antigen mixture is prepared in fine powder form and dispensed to the air space, in which a subject in need of the treatment lives or stays for part of his/her daily life. For example, the antigen mixture is supplied in the bedroom near the head of the bed where the subject sleeps, in the recreation room or study where the subject plays, rests, or studies, or in the office where the subject works. The antigen mixture can also be released to various rooms in a nursery, day-care center, or kindergarten, where a group of young children who need the treatment stay. When the antigen mixture is supplied in the air, the density of particle and the density of pollen proteins should mimic the outdoor density of pollens of common allergenic causes in an intense pollinating season.

When sterilized antigen mixtures collected from jungle areas are used in the treatment methods described herein, a subject in need of the treatment can be exposed to daily packs in a sequential manner, such as one pack per day for a suitable period (e.g., one year). Alternatively, the subject can be exposed to weekly packs, monthly packs, or seasonal packages also in a sequential manner, each package for a suitable period (e.g., one weekly pack per week for 52 weeks, one monthly pack per month for 12 months, or one seasonable pack per season for one year). In order to mimic the exposure to antigens in nature, the subject is recommended to be exposed to antigen packages in the same sequence the antigen particles are collected and in the same duration. In the native environment, the composition of the antigen particles changes gradually over each passing day. Therefore, the preferred method of applying the antigenic substances is to use the antigen packets in the order as they are collected.

When the immune packages described above that containing multiple preparations of characterized antigens are used for immune therapy, a subject in need of the treatment can be treated with the multiple preparations in the same manner as described above. The multiple preparations should contain antigens to which the subject has not been exposed to. In one example, the antigens are from areas where the subject has not lived for longer than two years.

In one example, the subject is exposed to the multiple preparations in an immune package in a sequential manner, each for a suitable period (e.g., 1 to 4 weeks). The total treatment period preferably lasts for several weeks (e.g., 4-12 weeks) to several years (e.g., 1-2 years). For example, the subject is exposed to 4, 8, or more preparations, each for several weeks (e.g. 4-12 weeks) to several months (e.g., 6-9 months).

If a subject is still allergic to local antigens after one course of treatment (e.g., with 4 preparations), the same treatment course can be repeated, using multiple preparations of antigens that are different from those used in the first course of treatment. If after the second course of treatment, the subject is still sensitive to local antigens, he/she probably has not produced sufficiently high amounts of IgE to the antigens of nonlocal origins. It is, therefore, safe for the patient to be exposed to the same set of preparations of allergen mix for another cycle of 4-period or 8-period course.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1

### Preparation and Characterization of Compositions Containing Antigen Mixtures Obtained from Primitive Jungles

A particle collector of the SASS 4000 or SASS 4100 model from Research International, Inc., Monroe, Wash., USA, is placed in a primitive jungle area, such as Xishuangbanna, Yunan Province, China. The particle collector is placed in a spot that is at least 20 km away from any human habitat. The particle collector is adjusted such that it collects airborne particles having a diameter ranging from 0.5 μm to 100 or 0.5 μm to 200 μm. Over 3500 liters/minute of ambient air are continuously collected.

Some types of the SASS series particle collectors collect particulates and water-soluble chemical vapors from the concentrated air and transfers them to a liquid phase. One to seven days after the particle collector is placed in the jungle area, the liquid phase is harvested and concentrated via a routine method to enrich the particles contained therein. The concentrated particles are then subjected to sucrose density gradient centrifugation to remove non-biological substances such as dirt and sand particles. The resultant compositions, which contain antigens collected from the jungle area (natural antigens), are sterilized by gamma or X-ray irradiation to produce a mixture of sterilized natural antigens. Another type of the SASS series particle collectors collect airborne particles in mesh filters in dry form and the trapped or attached particles are released or dissolved by aqueous solvent containing physiologically compatible detergent, such as surfactant.

A sample of the antigen mixture prepared as described above is subjected to routine biological assays to determine the complexity, identity, and relative proportions of the protein components contained in the samples. Briefly, the sample is treated with an aqueous solution containing sodium dodecyl sulfate or another equivalent detergent to break up the particles in the sample and release proteins contained therein into the aqueous solution. The number of detectable proteins, the identity of the major proteins, and the relative proportions of the major proteins are analyzed by a combination of polyacrylamide gel electrophoresis (PAGE), gas chromatography (GC), liquid chromatography (LC) (such as high pressure liquid chromatography, HPLC), and mass spectroscopy, e.g., electrospray ionization (ESI) and matrix-assisted laser desorption/ionization (MALDI). When the molecular weight and partial amino acid sequence of a protein are determined, the identity of that protein is determined by BLAST sequence analysis and comparison with known protein databases.

Immune cross-reactivity of the natural antigens in the composition prepared as described above is compared with pollen antigens from a different area as follows.

The natural antigen mixture is subjected to standard antigen extraction procedures to produce aqueous extracts containing the antigens. These extracts are analyzed by SDS-PAGE to determine their protein contents. They are further examined to determine primary allergens contained therein via routine analytical methods.

Cross-reactivity of the antigens in the extract with known pollen antigens is determined by standard ELISA analysis. Briefly, a panel of sera is obtained from patients who are allergic to identified pollen antigens as determined by standard skin prick tests. These patients do not live in places nearby the jungle area where the natural antigen mixture is collected. Their sera contain IgE antibodies specific to the identified pollen antigens. Natural antigens in the extracts, coated on a 96-well ELISA plate, are incubated with the panel of sera for a suitable period of time. After being washed to remove unbound antibodies, the plate is incubated with goat anti-human IgE antibodies conjugated with horseradish peroxidase or alkaline phosphatase as a second antibody for a suitable period. The plate is then subject to color development and colorimetric measurement to determine cross-reactivity of the natural antigens collected from the jungle area with the IgE antibodies in the patient sera, which are specific to identified pollen antigens derived from an area different from the jungle area.

### Example 2

### Multiple Antigen Preparations Containing Diversified Antigen Populations for Treating Allergy in Japanese Patients

Described herein is a package of four antigen preparations, each containing at least five different antigenic substances that have low or none reactivity with IgE antibodies in patients in Japan who are allergic to pollen antigens, dust mite antigens, cat antigens, and/or other local antigens. The predominant pollen allergens in Japan are derived from the pollens of Japanese cedar “sugi” (Cryptomeria japonica). Two allergenic proteins, cry j 1 and cry j 2, have been identified from sugi pollens. The five different antigenic substances in each preparation are in substantially equal amount; none of them is dominant in the preparation. The five antigenic substances in one preparation are different from those in another preparation.

The antigenic substances in the packages can be pollens from weeds and trees grown in areas outside Japan, e.g., pollens of trees that are distantly related to C. japonica or weed/tree pollens collected from tropical islands in the Pacific or Indian Oceans, from a geographical area in Africa, Central Asia, Philippines, Indonesia, India, Australia, or New Zealand.

Examples of pollens that have minimal cross-reactivity with local antigens in Japan, include pollens of Parkinsonia, Blackberry, Rubber vine, Parthenium, Lantana, Bitou bush, Gorse, Prickly acacia, Serrated tussock, Mimosa, Willows, Mesquite, Bridal creeper, Athel pine, Hymenachne, Cabomba, Salvinia, Alligator weed, Pond apple, and Chilean needle grass (Sinden et al., CRC for Australian Weed Management: The economic impact of weeds in Australia. 2004). The pollens of these weeds can be examined for cross-reactivity with the local allergenic antigens in Japan and those having low cross-reactivity are used for preparing the multiple packages.

The four preparations in the package are all formulated for intranasal administration. A patient in Japan who is found to be allergic to local pollens is exposed to the multiple preparations in a sequential manner, each for 4 weeks, via intranasal administration. If necessary, the patient can be treated with a second package containing multiple preparations of antigens, which are different from those that the patient has been exposed to.

### Example 3

### Anti-Allergy and Anti-Autoimmunity Effect of Natural Antigen Mixtures

Mice are sensitized to develop airway sensitivity or allergy-like symptoms to model allergen C. japonica antigen cry.j 1 ragweed antigen, Amb a 1, or dust mite antigen Der p 1., following the method described in Tsunematsu et al., “A new murine model of allergic rhinitis by repeated intranasal Cry j 1 challenge” Biomed Res. 29:119-123 (2008). The mice thus treated develop airway inflammation and increased release of inflammatory cytokines, which can be detected in the blood. The mice are then exposed to a composition containing natural antigens as described in Example 1 above using a nebulizer. Mice exposed to a placebo (containing to antigenic substances) are used as blank controls. After being treated for a suitable period, the mice are challenged with the sensitizing antigen cry.j 1, Amb a 1, or Der p 1 and examined to determine their immune responses to the sensitizing antigens, e.g., levels of antigen-specific IgE antibodies and inflammatory cytokines. The anti-allergy effects of the natural antigen-containing compositions are evaluated accordingly.

Alternatively, male DBA/1 mice are injected intradermally at the base of their tails with 100 μg type-II collagen, emulsified in Freund complete adjuvant. Type II collagen is the major constituent of articular cartilage. The treated mice produce anti-collagen antibodies, increase secretion of tumor necrosis factor-1, interleukin-1, and develop swollen joints, such as thickened paws, all of which resemble symptoms of rheumatoid arthritis in human patients. These various biological parameters and pathological manifestations developed in the collagen-treated mice are assessed quantitatively. The mice with arthritic symptoms are exposed to the natural antigen-containing compositions described in Example 1 above. Mice treated with a placebo are used as a negative control. The effects of the exposure to natural antigen mix in alleviating arthritic symptoms are then evaluated.

### Example 4

### Enhancing IgE Production in Mice Using Natural Antigen Mixtures

Initial steps were taken to analyze the relative amounts and complexity (or diversity) of airborne protein substances collected from different locations and at different times and to formulate the routine procedures for collecting air particles and manipulating them. Those analyses were fundamental to the present disclosure.

A SASS 3100 particle collector from Research International and a large number of filters were used in this study. SASS 3100 is a smaller unit of the SASS series of particle collectors and can be powered by batteries and operated in remote sites, which are not adjacent to any electrical power outlets. Both the SASS 3100 unit and the batteries are easily hand-carried. The SASS 3100 unit collects 300 liters of air per minute.

The SASS 3100 collector was set up with an air particle collection procedure allowing the machine to run for 24 hours. The opening of the air inlet of the collector was covered by a screen with a mesh of 500 μm to prevent from the suction of large particles. The geographic sites chosen for analyses and comparison include the bedroom in a residential house in Taipei, the office of the principal investigator and research laboratory in the Genomics Research Center, Academia Sinica, Taipei, Taiwan, the covered outdoor hallway of the Genomics Research building, the wooded garden Yuan-Pei Park inside the Academia Sinica campus, and a primitive, bio-diverse Fu-Shan National Park in I-Lan County on the East Coast of Taiwan Island. It is noted that the residential house, which was located in the center city of Taipei, did not use air conditioning, while the Genomics Research Center was under air conditioning almost all time. The campus of Academia Sinica is located on the outskirt of Taipei City, near a mountainous area. The collection procedures were repeated at different times at the same locations.

The particles collected on the filter, which was 4.3 cm in diameter and about 2 mm in thickness, were immersed and dissolved in 3 ml phosphate-buffered saline (PBS) supplemented with 0.1% Tween 20 detergent at 37° C. for 1 hour. In certain procedures, the filters were again soaked with 3 ml PBS (with 0.1% Tween 20) at 37° C. for 24 hours. It was observed that most of the protein substances captured by the filter was released in the first 1-hour wash/soaking procedure with PBS (0.1% Tween 20). The protein contents were subjected to 13% SDS PAGE and stained with Pierce Silver Stain Kit (Thermo Scientific, Rockville, Ill., USA).

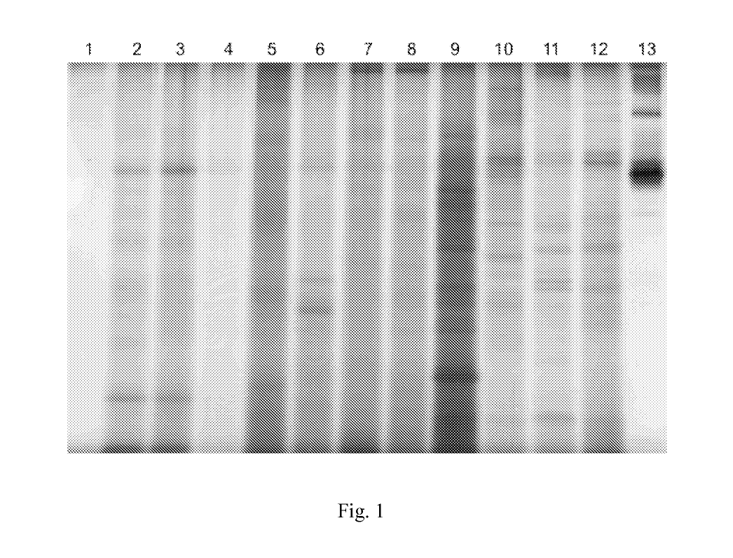

The gel analytical patterns of certain samples collected as described above were exemplified by the results shown in FIG. 1. It has been observed that: (1) the outdoor protein antigens in general were much more diverse than indoor protein antigens, (2) in an interior space with air conditioning, the protein substances were sparse and the diversity limited, (3) the protein substances collected in different days, even only two-weeks apart, showed rather distinct patterns and revealed different total protein amounts, (4) the protein substances collected in the primitive Fu-Shan National Park showed the most diverse, heterogeneous composition and no dominant bands. These results support the fundamental concept disclosed herein.

In order to test the effect of natural antigen mixtures, a series of experiments were performed using Balb/c mice to establish the procedure for sensitizing mice intra-nasally with the collected airborne protein substance. In one typical set of experiments, the PBS (0.1% Tween 20) solution of particles collected in Fu-Shan National Park, which was prepared as described above, was further diluted 3 times and 20 times and used in the nasal stimulation of mice. Blank solution (PBS with 0.1% Tween 20) and ovalbumin at 80 μg/ml in the same solution were used as controls.

In one experiment, 20 Balb/c mice were divided into 4 groups, 5 animals in each group, and each group received one of the 4 samples for nasal stimulation. Each Balb/c mouse was anesthetized by intraperitoneal injection of 130 μl of 10-fold diluted Zoletil 50 (Virbac Animal Health, Carros, France). Each of the animals was then administered with 20 μl of the protein antigens intra-nasally. This procedure was performed daily for 4 weeks or longer. Blood samples were obtained from the animals by submandibular bleeding before and after the immunization procedure weekly. The total serum IgE was analyzed by an ELISA quantification set (Bethyl Laboratories, Montgomery, Tex., USA). The ELISA for testing IgE antibodies induced by the injected proteins was also been established.

The data indicated that the protein mixture present in the airborne air particles from Fu-Shan National Park, as well as ovalbumin, elicited small increases in total IgE by 28 days, indicating that the natural protein mixtures collected as described herein could be effective in enhancing IgE production in a subject. Alonger period of administration is predicted to have better effects.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A composition comprising a mixture of sterilized antigens, wherein the mixture of sterilized antigens is prepared by a process comprising:
placing a particle collector in a jungle area,
harvesting airborne antigens collected in the particle collector, and
sterilizing harvested airborne antigens to obtain the mixture of the sterilized antigens.

2. The composition of claim 1, wherein the process further comprises, prior to sterilizing, removing non-biological substances from the harvested airborne antigens.

3. The composition of claim 2, wherein the non-biological substances are removed by density gradient centrifugation.

4. The composition of claim 1 wherein the particle collector collects substantially only airborne antigens having a diameter ranging from about 0.5 μm to about 200 μm.

5. The composition of claim 4, wherein the particle collector collects substantially only airborne antigens having a diameter ranging from about 0.5 μm to about 150 μm.

6. The composition of claim 5, wherein the particle collector collects substantially only airborne antigens having a diameter ranging from about 0.5 μm to about 100 μm.

7. The composition of claim 1 wherein harvesting airborne antigens from the particle collector is carried out 1 to 7 days after the particular collector was placed in the jungle area.

8. The composition of claim 1 wherein harvesting airborne antigens from the particle collector is carried out once every 1 to 7 days for up to twelve months and the harvested airborne antigens are combined.

9. The composition of claim 1 wherein the sterilizing step is performed by gamma ray or X ray irradiation.

10. The composition of claim 1 wherein the composition is formulated for non-invasive administration.

11. The composition of claim 10, wherein the non-invasive administration is intranasal administration, sublingual administration, or oral administration.

12. The composition of claim 1 wherein the composition is a pharmaceutical formulation, a food product, or a dietary supplement.

13. The composition of claim 1 wherein the composition is for use in enhancing production of IgE antibodies.

14. The composition of claim 1 wherein the composition is for use in treating an allergic disease.

15. The composition of claim 14, wherein the allergic disease is allergic asthma, allergic rhinitis, atopic dermatitis or food allergy.

16. The composition of claim 1 wherein the composition is for use in treating an autoimmune disease.

17. The composition of claim 16, wherein the autoimmune disease is rheumatoid arthritis, systemic lupus erythematosus, multiple sclerosis, autoimmune thyroid disease, Type I diabetes, or inflammatory bowel disease.

18. A kit comprising multiple preparations of sterilized antigen mixtures, each of the preparations being prepared by a process comprising:
placing a particle collector in a jungle area,
harvesting airborne antigens collected in the particle collector, and
sterilizing harvested airborne antigens to obtain a preparation of sterilized antigen mixture; wherein the multiple preparations of sterilized antigen mixtures are obtained from geographically different jungle areas or from the same jungle area but during different time periods.

19. The kit of claim 18, wherein the process further comprising, prior to sterilizing, removing non-biological substances from the harvested airborne antigens.

20. The kit of claim 19, wherein the non-biological substances are removed by density gradient centrifugation.

21. The kit of claim 18 wherein the particle collector collects substantially only airborne antigens having a diameter ranging from about 0.5 μm to about 200 μm.

22. The kit of claim 21, wherein the particle collector collects substantially only airborne antigens having a diameter ranging from about 0.5 μm to about 150 μm.

23. The kit of claim 22, wherein the particle collector collects substantially only airborne antigens having a diameter ranging from about 0.5 μm to about 100 μm.

24. The kit of claim 18 wherein harvesting airborne antigens from the particle collector is carried out 1 to 7 days after the particular collector was placed in the jungle area.

25. The kit of claim 18 wherein harvesting airborne antigens from the particle collector is carried out once every 1 to 7 days for up to twelve months.

26. The kit of claim 25, wherein one or more of the multiple preparations contain daily harvested airborne antigens.

27. The kit of claim 25, wherein one or more of the multiple preparations contain airborne antigens harvested during one week.

28. The kit of claim 18 wherein the sterilizing step is performed by gamma ray or X ray irradiation.

29. The kit of claim 18 wherein the multiple preparations are formulated for non-invasive administration.

30. The kit of claim 29, wherein the non-invasive administration is intranasal administration, sublingual administration, or oral administration.

31. The kit of claim 18 wherein the multiple preparations are pharmaceutical formulations, food products, or dietary supplements.

32. The kit of claim 18 wherein the multiple preparations are for use in enhancing production of IgE antibodies.

33. The kit of claim 18 wherein the multiple preparations are for use in treating an allergic disease.

34. The kit of claim 33, wherein the allergic disease is allergic asthma, allergic rhinitis, atopic dermatitis, or food allergy.

35. The kit of claim 18 wherein the composition is for use in treating an autoimmune disease.

36. The kit of claim 35, wherein the autoimmune disease is rheumatoid arthritis, systemic lupus erythematosus, multiple sclerosis, Type I diabetes, autoimmune thyroid disease, or inflammatory bowel disease.

37. A kit comprising multiple preparations of sterilized antigen mixtures, each of the preparations containing at least five sterilized natural antigens, wherein:
(a) the amount of each antigen in each preparation is effective in inducing IgE antibody production, and
(b) at least 20% of the natural antigens in one preparation are not present in any other preparations in the kit.

38. The kit of claim 37, wherein the natural antigens are weed pollens, tree pollens, mold antigens, microorganism antigens, or worm antigens.

39. The kit of claim 37 wherein the natural antigens are geographically specific.

40. The kit of claim 37 wherein each of the natural antigens in the multiple preparations is sterilized by gamma ray or X ray irradiation.

41. The kit of claim 37 wherein the multiple preparations are formulated for non-invasive administration.

42. The kit of claim 41, wherein the non-invasive administration is intranasal administration, sublingual administration, or oral administration.

43. The kit of claim 37 wherein the multiple preparations are pharmaceutical formulations, food products, or dietary supplements.

44. The kit of claim 37 wherein the multiple preparations are for use in enhancing production of IgE antibodies.

45. The kit of claim 37 wherein the multiple preparations are for use in treating an allergic disease.

46. The kit of claim 45, wherein the allergic disease is allergic rhinitis, or food allergy, atopic dermatitis, or allergic asthma.

47. The kit of claim 37 wherein the composition is for use in treating an autoimmune disease.

48. The kit of claim 47, wherein the autoimmune disease is rheumatoid arthritis, systemic lupus erythematosus, multiple sclerosis, Type I diabetes, autoimmune thyroid disease, or inflammatory bowel disease.

49. The kit of claim 37 wherein the kit comprises at least 4 preparations.

50. A method for preparing a composition, comprising:
placing a particle collector in a jungle area,
harvesting airborne antigens collected in the particle collector, and
sterilizing the airborne antigens to obtain a composition, which comprises a mixture of sterilized antigens.

51. The method of claim 50, further comprising, prior to sterilizing, removing non-biological substances from the harvested airborne antigens.

52. The method of claim 51, wherein the non-biological substances are removed by density gradient centrifugation.

53. The method of claim 50 wherein the particle collector collects substantially only airborne antigens having a diameter ranging from about 0.5 μm to about 200 μm.

54. The method of claim 53, wherein the particle collector collects substantially only airborne antigens having a diameter ranging from about 0.5 μm to about 150 μm.

55. The method of claim 54, wherein the particle collector collects substantially only airborne antigens having a diameter ranging from about 0.5 μm to about 100 μm.

56. The method of claim 50 wherein harvesting airborne antigens from the particle collector is carried out 1 to 7 days after the particular collector was placed in the jungle area.

57. The method of claim 50 wherein harvesting airborne antigens from the particle collector is carried out once every 1 to 7 days for up to twelve months and at least a portion of the harvested airborne antigens are combined.

58. The method of claim 50 wherein harvesting airborne antigens from the particle collector is carried out every day for up to twelve months and the antigens harvested each day is packed individually to produce daily packs, combined with others to produce weekly packs, monthly packs, or seasonal packs.

59. The method of claim 50 wherein the sterilizing step is performed by gamma ray or X ray irradiation.

60. The method of claim 50 further comprising formulating the mixture of sterilized antigens to a pharmaceutical formulation, a food product, or a dietary supplement.

61. A method for enhancing IgE production in a subject, comprising administering to a subject in need thereof an effective amount of a composition of claim 1.

62. The method of claim 61, wherein the composition is administered to the subject via a non-invasive route.

63. The method of claim 62, wherein the non-invasive route is exposing the subject to the composition by releasing the composition to a room where the subject stays.

64. The method of claim 62, wherein the non-invasive route is intranasal administration, sublingual administration, or oral administration.

65. The method of claim 61 wherein the subject is a human patient having, suspected of having, or at risk for an allergic disease.

66. The method of claim 65, wherein the allergic disease is allergic asthma, allergic rhinitis, atopic dermatitis, or food allergy.

67. The method of claim 61 wherein the subject is a human patient having, suspected of having, or at risk for an autoimmune disease.

68. The method of claim 67, wherein the autoimmune disease is rheumatoid arthritis, systemic lupus erythematosus, multiple sclerosis, Type I diabetes, autoimmune thyroid disease, or inflammatory bowel disease.

69. A method for enhancing IgE production in a subject, comprising exposing a subject in need thereof multiple preparations of sterilized antigen mixtures in a kit of claim 18 wherein the subject is exposed to the multiple preparations sequentially.

70. The method of claim 69, wherein the subject is exposed to the sterilized antigen mixtures in each of the multiple preparations for 1 to 4 weeks.

71. The method of claim 69 wherein the subject is exposed to the multiple preparations via a non-invasive route.

72. The method of claim 71, wherein the non-invasive route is intranasal administration, sublingual administration, or oral administration.

73. The method of claim 71, wherein the non-invasive route is exposing the subject to the composition by releasing each of the multiple preparation to a room where the subject stays.

74. The method of claim 69 wherein the subject is a human patient having, suspected of having, or at risk for an allergic disease.

75. The method of claim 74, wherein the allergic disease is allergic asthma, allergic rhinitis, atopic dermatitis, or food allergy.

76. The method of claim 69 wherein the subject is a human patient having, suspected of having, or at risk for an autoimmune disease.

77. The method of claim 76, wherein the autoimmune disease is rheumatoid arthritis, systemic lupus erythematosus, multiple sclerosis, Type I diabetes, autoimmune thyroid disease, or inflammatory bowel disease.

78. A method for enhancing IgE production in a subject, comprising administering to a subject in need thereof at least four multiple preparations of sterilized antigen mixtures sequentially, wherein
(a) each of the preparations contains at least five sterilized natural antigens,
(b) the amount of each antigen in each preparation is effective in inducing IgE antibody production,
(c) at least 20% of the natural antigens in one preparation are not present in any other preparations in the kit, and
(d) the natural antigens in the multiple preparations are obtained from a place in which the subject has not lived for longer than 2 years.

79. The method of claim 78, wherein the subject is exposed to each preparation for 1 to 4 weeks.

80. The method of claim 78 wherein the natural antigens are weed pollens, tree pollens, mold antigens, microorganism antigens, or worm antigens.

81. The method of claim 78 wherein the natural antigens are geographically specific.

82. The method of claim 78 wherein each of the natural antigens in the multiple preparations is sterilized by gamma ray or X ray irradiation.

83. The method of claim 79 wherein subject is exposed to the multiple preparations by a non-invasive route.

84. The method of claim 83, wherein the non-invasive route is exposing the subject to the composition by releasing each of the multiple preparations to a room where the subject stays.

85. The method of claim 83, wherein the non-invasive route is intranasal administration, sublingual administration, or oral administration.

86. The method of claim 78 wherein the multiple preparations are pharmaceutical formulations, food products, or dietary supplements.

87. The method of claim 78 wherein the subject is a human patient having, suspected of having, or at risk for an allergic disease.

88. The method of claim 87, wherein the allergic disease is allergic asthma, allergic rhinitis, atopic dermatitis, or food allergy.

89. The method of claim 78 wherein the subject is a human patient having, suspected of having, or at risk for an autoimmune disease.

90. The method of claim 89, wherein the autoimmune disease is rheumatoid arthritis, systemic lupus erythematosus, multiple sclerosis, Type I diabetes, autoimmune thyroid disease, or inflammatory bowel disease.
